(19) United States
(12) Patent Application Publication
(10) Pub. No.: US 2007/0154499 A1
(43) Pub. Date: Jul. 5, 2007

(54) Masculine homeopathic personal lubricant and method of use

(76) Inventor: Jason Fleming, Trinidad, CA (US)

Correspondence Address: Jason Fleming, 228 Commercial St. #287, Nevada City, CA, 95959, US

(21) Appl. No.: 11/328,467

(22) Filed: Jan. 5, 2006

(51) Int. Cl. A61K 8/02

(52) U.S. Cl. 424/401

(57) ABSTRACT

The personal lubricants of the present invention are lubricants, preferably made from substantially wholly organic ingredients, which include homeopathic ingredients which are known to be useful to support the male reproductive system and to support the strength of the male erection and increase sexual stamina. The lubricants are preferably water-based. In a preferred embodiment the lubricants are rehydrating.

### TECHNICAL FIELD

This invention relates to lubricants, and more particularly to personal lubricants useful for sexual intercourse and methods for their use. The lubricants are water-based and contain natural, organic ingredients, including homeopathic ingredients useful in supporting the male reproductive system.

### BACKGROUND

The use of personal lubricants as an aid to sexual intercourse is known in the art. However, previous personal lubricant compositions were generally based on petroleum distillates and other non-organic components which can cause adverse reactions such as allergies or irritate sensitive tissues.

Homeopathic remedies are known to restore balance in the body and stimulate the natural healing power of the body. See, e.g., Homeopathic Remedy Guide by Robin Murphy ND (2000) and Homeopathic Medical Repertory by Robin Murphy ND Second Edition (2003). Generally, homeopathic remedies are administered orally to neutralize disorders, including disorders relating to male and female sexuality.

Intimate contact between individuals, such as during sexual intercourse, often suffers from an undesirably high amount of friction between the individuals. It is known in the prior art to provide various personal lubricants to reduce the coefficient of friction exhibited between the individuals to enhance the comfort and pleasure associated with the experience. One common category of such lubricants are lubricants formed from petroleum distillates, such as glycerine mixed with a water base. Various products are known to be provided in this category, which typically include various other ingredients in their particular formulations to distinguish them from each other.

Glycerine is known to be an effective lubricant and to be water soluble and generally acceptable for use directly on human skin. However, this and other petroleum distillate derived personal lubricants, such as other glycerine based lubricants, suffer from numerous drawbacks.

Specifically, such products are not organically derived, but rather manufactured through chemical engineering techniques, typically from crude oil or other petroleum products. Thus, such products suffer from all of the known, and potential unknown consequences resulting from the use of such products. Many individuals and experts have come to recognize that when products are provided which are derived from organic sources, rather than manufactured from petroleum products, that the effectiveness of such products is enhanced.

Further, oil-containing personal lubricants can cause rapid deterioration of latex condoms. As little as one minute's exposure to mineral oil caused approximately 90% decrease in the strength of condoms. (Voeller et al., Contraception 39:95-102 (1989)) Additionally, some individuals can experience allergies or other undesirable reactions when utilizing products distilled from petroleum while not having corresponding detrimental reactions to organically derived products. Finally, many individuals recognize the benefits of the lesser impact on the environment and the benefits of sustainable develop associated with organic products. While these benefits alone suggest the desirability for the providing of personal lubricants made from materials other than petroleum distillates, such petroleum distillate lubricants also provide a less than wholly satisfactory function as a personal lubricant.

Particularly, these petroleum distillate personal lubricants experience particular negative attributes in many circumstances. Such petroleum distillate personal lubricants can experience a dehydration phenomena where, as the petroleum distillate personal lubricant formula undergoes evaporation or selective absorption such that the lubricant begins to dry out, the lubricant undergoes a radical increase in coefficient of friction, changing from slick to sticky. This phenomenon can be particularly recognized by merely placing petroleum distillate based personal lubricants on ones hands and rubbing ones hands together for about two minutes. The petroleum distillate formulation will dry out and become sticky, actually exacerbating the friction problem rather than remediating it. When water is added to the water based petroleum distillate personal lubricants they do return from sticky to slick. While this return to slickness does last for a few minutes, further dehydration causes the personal lubricant to again attain a sticky character.

Further rehydration fails-to renew the petroleum distillate based personal lubricants. Rather, they attain a coefficient of friction similar to that of water when sufficient rehydration has occurred, such that the petroleum distillate based personal lubricants lose their effectiveness after about five minutes. Often such a minimal duration of function is unacceptable as the period of intimate contact has not yet ceased, resulting in discomfort or disruption for reapplication of additional lubricant.

Additionally, lack of male stamina or erectile strength during intercourse may result in a less satisfactory experience for one or both partners. Prior art lubricants do not address this problem by the use of homeopathic ingredients which can improve male stamina and/or erectile strength.

Accordingly, a need exists for a personal lubricant which is derived from organic sources and contains homeopathic ingredients which can provide enhanced health benefits. In particular, a homeopathic-containing personal lubricant which is derived from organic sources rather than from petroleum distillates and maintains its slipperiness for as long a duration as possible and which is readily rehydratable with liquids secreted by the individuals during intercourse, such that slipperiness is maintained for a long period of time without requiring rehydration would be desirable.

### SUMMARY

The personal lubricants of the present invention are lubricants, preferably made from substantially wholly organic ingredients, which include homeopathic ingredients which are known to be useful to support the male reproductive system and to support the strength of the male erection and increase sexual stamina. The lubricants are preferably water-based. In a preferred embodiment the lubricants are rehydrating, as described in U.S. patent application Ser. No. 10/795,172, filed Mar. 4, 2004, the disclosure of which is incorporated herein by reference in its entirety. Such lubricants contain guar gum and exhibit native rehydration when in use. The compositions and formulations of the present invention have a low coefficient of friction when applied to mating surfaces, such that they function effectively as lubricants and especially as personal lubricants such as for sexual intercourse.

Accordingly, in one aspect the invention provides a personal lubricant composition comprising at least one component selected from the group consisting of lycopodium, testicle, sepia, hypericum, agnus castus, kidney, petroselinum sativum, liver, cantharis, berberis vulgaris and solidago vigaurea and combinations thereof. The lubricant may be water-based. The lubricant may preferably be substantially all natural and/or all organic. In particular, lubricants containing at least one of lycopodium, testicle, sepia, agnus castus, petroselinum sativum and combinations thereof are useful to support the male reproductive system.

In another aspect, the invention provides a rehydrating water-based personal lubricant composition comprising gum and at least one component selected from the group consisting of lycopodium, testicle, sepia, hypericum, agnus castus, kidney, petroselinum sativum, liver, cantharis, berberis vulgaris and solidago vigaurea. The lubricant may preferably be substantially all natural and/or all organic. In particular, lubricants containing at least one of lycopodium, testicle, sepia, agnus castus, petroselinum sativum and combinations thereof are useful to support the male reproductive system.

In yet another aspect, the invention provides a rehydrating water-based personal lubricant composition comprising guar gum, at least one component selected from material derived from an aloe vera plant, grapefruit seed extract and vitamin E, and at least one component selected from the group consisting of lycopodium, testicle, sepia, hypericum, agnus castus, kidney, petroselinum sativum, liver, cantharis, berberis vulgaris and solidago vigaurea. The lubricant may preferably be substantially all natural and/or all organic. In particular, lubricants containing at least one of lycopodium, testicle, sepia, agnus castus, petroselinum sativum and combinations thereof are useful to support the male reproductive system.

In yet a further aspect, the invention provides a rehydrating water-based personal lubricant composition comprising material derived from an aloe vera plant, grapefruit seed extract, vitamin E, guar gum and at least one component selected from the group consisting of lycopodium 6c, testicle 4c, sepia 6c, hypericum 6c, agnus castus 6c, kidney 9c, petroselinum sativum 6c, liver 9c, cantharis 6c, berberis vulgaris 6c and solidago vigaurea 6c. The lubricant may preferably be substantially all natural and/or all organic. In particular, lubricants containing at least one of lycopodium 6c, testicle 4c, sepia 6c, agnus castus 6c, petroselinum sativum 6c and combinations thereof are useful to support the male reproductive system.

In still another aspect, the invention provides methods of using the personal lubricant compositions described above by applying the lubricant to a male individual prior to sexual intercourse.

The details of one or more embodiments of the invention are set forth in the description below. Other features, objects, and advantages of the invention will be apparent from the description, and from the claims.

### DETAILED DESCRIPTION

This invention is directed to personal lubricants useful for sexual intercourse which support the male reproductive system. The lubricants are preferably water-based and made from substantially wholly organic ingredients. They include homeopathic ingredients known to be useful to support the male reproductive system and to support the strength of the male erection and increase sexual stamina. In a preferred embodiment the lubricants are rehydrating, as described in U.S. patent application Ser. No. 10/795,172, filed Mar. 4, 2004, the disclosure of which is incorporated herein by reference in its entirety, which describes lubricants containing guar gum and exhibiting native rehydration when in use. The compositions and formulations of the present invention have a low coefficient of friction when applied to mating surfaces, such that they function effectively as lubricants and especially as personal lubricants such as for sexual intercourse. In addition, they contain homeopathic components which support the strength of the male erection and increase sexual stamina.

The personal lubricants of the present invention comprise homeopathic ingredients useful to support the male reproductive system in a lubricant base. A water-based lubricant is preferred, with rehydrating water-based lubricants being especially preferred. In one embodiment, the rehydrating water-based personal lubricant composition comprises material derived from an aloe vera plant, grapefruit seed extract, vitamin E and guar gum. See U.S. patent application Ser. No. 10/795,172, filed Mar. 4, 2004.

The water-based personal lubricant compositions of the present invention also comprise at least one homeopathic component selected from the group consisting of lycopodium, testicle, sepia, hypericum, agnus castus, kidney, petroselinum sativum, liver, cantharis, berberis vulgaris and solidago vigaurea and combinations thereof. These homeopathic ingredients may be purchased, for example, from Dolisos Homeopathic Remedies. (http://www.zhealthinfo.com/dolisos.htm) Various strengths may be used, but the preferred strength of these homeopathic components is: lycopodium 6c, testicle 4c, sepia 6c, hypericum 6c, agnus castus 6c, kidney 9c, petroselinum sativum 6c, liver 9c, cantharis 6c, berberis vulgaris 6c and solidago vigaurea 6c. In particular, it has been found that lubricants containing at least one of lycopodium, testicle, sepia, agnus castus, petroselinum sativum and combinations thereof are especially useful to support the male reproductive system.

Each homeopathic component used may be present in the lubricant composition in an amount ranging from about 0.0001% to about 10%, by volume. Certain embodiments may contain from about 0.001% to about 1% (by volume) of each homeopathic component that is present in the personal lubricant composition. In further embodiments, a homeopathic component may comprise from about 0.01% to about 0.1% (v/v) of the personal lubricant composition. In one embodiment, about 0.02% to about 0.03% by volume of each homeopathic component is used.

All possible combinations of the homeopathic components may be used in the lubricants of the present invention. That is to say, a personal lubricant of the present invention may contain one, two, three, four, five, six, seven, eight, nine, ten or eleven of the homeopathic components lycopodium, testicle, sepia, hypericum, agnus castus, kidney, petroselinum sativum, liver, cantharis, berberis vulgaris and solidago vigaurea, in any combination.

The constituents which make up the water-based rehydrating personal lubricant base useful in this invention according to its preferred embodiments are guar gum and water or other water containing solvent sufficient to cause the guar gum to form a gel. The water containing solvent is preferably deionized water. The ratio of guar gum to water containing solvent is preferably between about 1% and about 2%. However, if the amount of the formulation provided by guar gum is between about 0.01% and about 15%, many of the benefits of this invention can be achieved, with formulations having an amount of guar gum between about 1.0% and about 10% considered to be within a most desirable range.

Many of the benefits of the personal lubricant of this invention can be provided by merely combining the guar gum with a water containing solvent as identified above. The water containing solvent can be water alone, and preferably deionized water. This water containing solvent can also be aloe vera juice or a combination of aloe vera juice and water. This water containing solvent can also be other water containing solvents capable of converting the guar gum from a paste consistency into a gel.

The composition derives further benefits from including other constituents as follows. The composition preferably includes both water in the form of deionized water and material from the aloe vera plant, most preferably in the form of aloe vera juice. Aloe vera juice is particularly provided by filleting the leaves of the aloe vera plant and squeezing/pressing them to extract the “juice.” The aloe vera juice is preferably provided in a range between about 0% and about 90%. Any amount between about 70% and about 90% is considered most desirable, with about 80% considered to be optimal.

The preferred formulation additionally preferably includes grapefruit seed extract. This grapefruit seed extract is preferably provided as between about 0.001% and about 5% of the entire formulation. Most desirably the grapefruit seed extract is provided in an amount between about 0.01% andabout 0.5% with about 0.10% to about 0.15% considered most optimal.

The preferred formulation preferably includes vitamin E as a natural preservative. The vitamin E additionally provides associated health benefits known to be associated with vitamin E. The vitamin E is preferably provided as between about 0% and about 5% of the entire formulation. Amounts of vitamin E between about 1.0% and about 3.0% are considered most desirable, with about 2.3% to about 2.8% considered optimal.

Additional constituents can additionally be provided within the formulation if desired, such as flavorings, fragrances, aromas, medicinals, colorants, etc.

The process of mixing the constituents together preferably entails combining first the ingredients of the water-based lubricant and the homeopathic components. Food grade equipment and conditions are used in the preparation of the lubricant compositions.

In the case of the rehydrating personal lubricant compositions described above, generally, the deionized water and organic aloe vera juice are combined, then vitamin E is added as a natural preservative, then flavor is added (if included), then grapefruit seed extract is added and finally guar gum is added. The other constituents, e.g., the homeopathic ingredients, can be added at various different times. Everything is mixed in a food grade container. After the mix turns to gel, it is pumped into a hopper which can conveniently feed it into bottles or other sealed containers for later distribution and use.

The invention also provides methods of using the personal lubricant compositions described above by applying the lubricant to a male individual prior to sexual intercourse. The homeopathic components of the lubricant serve to strengthen the erection and provide greater stamina of the male individual.

When the rehydratable formulation is utilized during sexual intercourse it is effectively rehydrated with the liquids secreted by the individuals involved. Such native rehydration has been experienced which has provided a nearly unlimited duration of high degree of slipperiness with the use of this formulation of this invention. In contrast, similar use of personal lubricants derived from petroleum distillates has not been shown to be benefited particularly from any rehydration with bodily secreted liquids. Hence, this native rehydration effect, which provides significant additional duration to the effectiveness of the lubrication, has been found to be uniquely present with the formulation of this invention, and absent from prior art formulations such as those derived from petroleum distillates.

In use and operation, this formulation of this invention is preferably used as follows. The formulation is initially provided in a container which isolates the formulation from the surrounding environment. Typically, a bottle is provided from which the formulation can be squeezed in a relatively controllable fashion. The users then apply the formulation to at least one of the surfaces of one body part of a male individual which is to be involved in the contact between the individuals. As an acceptable alternative, the formulation can be applied to multiple body parts of the male individual. After direct application has occurred, contact between the individuals is initiated with the lubricant located between the individuals.

As an optional intermediate step, a condom or other additional device can be interposed between the individuals.

After contact is initiated, such contact will typically stimulate the secretion of bodily liquids. When a rehydrating lubricant formula is used, these bodily liquids interact with the formulation in a fashion which rehydrates the formulation and enhances a slipperiness of the formulation by reducing a coefficient of friction exhibited by the formulation. Such rehydration utilizing bodily secreted liquids is referred to as native rehydration. This native rehydration continues to maintain a low coefficient of friction and a high degree of slipperiness of the personal lubricant while the contact between the individuals continues. As a result, a high degree of durational effectiveness is maintained. The individuals thus need not interrupt the activity to apply additional lubricant as frequently or require external sources of water or other rehydrating agents to maintain the slipperiness of the personal lubricant. If the individuals secrete an insufficient amount of liquid for effective native rehydration, the individuals can optionally provide water from a separate source to rehydrate the personal lubricant formulation of this invention.

When the activity ceases, the formulation is easily removed by merely washing off, as the formulation is water based.

This disclosure is provided to reveal a preferred embodiment of the invention and a best mode for practicing the invention. Having thus described the invention in this way, it should be apparent that various different modifications can be made to the preferred embodiment without departing from the scope and spirit of this disclosure.

### EXAMPLE 1

After having a prostate procedure, a 73 year old male individual had trouble getting erections. His doctor prescribed a medical pump to help him achieve erection, which was uncomfortable and, according to the subject, didn't work very well. The subject used the rehydrating water-based personal lubricant composition comprising material derived from an aloe vera plant, grapefruit seed extract, vitamin E, guar gum, lycopodium 6c, testicle 4c, sepia 6c, hypericum 6c, agnus castus 6c, kidney 9c, petroselinum sativum 6c, liver 9c, cantharis 6c, berberis vulgaris 6c and solidago vigaurea 6c, applied as described above. The subject reported that use of the personal lubricant helped bring his natural erections back.

A number of embodiments of the invention have been described. Nevertheless, it will be understood that various modifications may be made without departing from the spirit and scope of the invention. Accordingly, other embodiments are within the scope of the following claims.

What is claimed is:

1. A water-based personal lubricant composition comprising at least one component selected from the group consisting of lycopodium, testicle, sepia, hypericum, agnus castus, kidney, petroselinum sativum, liver, cantharis, berberis vulgaris and solidago vigaurea.

2. The composition of claim 1 which contains at least two components.

3. The composition of claim 1 which contains at least three components.

4. The composition of claim 1 which contains at least four components.

5. The composition of claim 1 which contains at least five components.

6. The composition of claim 1 which contains at least six components.

7. The composition of claim 1 which contains at least seven components.

8. The composition of claim 1 which contains at least eight components.

9. The composition of claim 1 which contains at least nine components.

10. The composition of claim 1 which contains at least ten components.

11. The composition of claim 1 which contains at least eleven two components.

12. The composition of claim 1 which comprises at least one component selected from the group consisting of lycopodium, testicle, sepia, agnus castus and petroselinum sativum.

13. The composition of claim 1 which comprises substantially all natural and/or all organic ingredients and components.

14. A rehydrating water-based personal lubricant composition comprising at least one component selected from the group consisting of lycopodium, testicle, sepia, hypericum, agnus castus, kidney, petroselinum sativum, liver, cantharis, berberis vulgaris and solidago vigaurea.

15. The composition of claim 14 which contains at least two components.

16. The composition of claim 14 which contains at least three components.

17. The composition of claim 14 which contains at least four components.

18. The composition of claim 14 which contains at least five components.

19. The composition of claim 14 which contains at least six components.

20. The composition of claim 14 which contains at least seven components.

21. The composition of claim 14 which contains at least eight components.

22. The composition of claim 14 which contains at least nine components.

23. The composition of claim 14 which contains at least ten components.

24. The composition of claim 14 which contains at least eleven two components.

25. The composition of claim 14 which comprises at least one component selected from the group consisting of lycopodium, testicle, sepia, agnus castus and petroselinum sativum.

26. The composition of claim 14 which comprises substantially all natural and/or all organic ingredients and components.

27. A rehydrating water-based personal lubricant composition comprising guar gum and at least one component selected from the group consisting of lycopodium, testicle, sepia, hypericum, agnus castus, kidney, petroselinum sativum, liver, cantharis, berberis vulgaris and solidago vigaurea.

28. The composition of claim 27 which contains at least two components.

29. The composition of claim 27 which contains at least three components.

30. The composition of claim 27 which contains at least four components.

31. The composition of claim 27 which contains at least five components.

32. The composition of claim 27 which contains at least six components.

33. The composition of claim 27 which contains at least seven components.

34. The composition of claim 27 which contains at least eight components.

35. The composition of claim 27 which contains at least nine components.

36. The composition of claim 27 which contains at least ten components.

37. The composition of claim 27 which contains at least eleven two components.

38. The composition of claim 27 which comprises at least one component selected from the group consisting of lycopodium, testicle, sepia, agnus castus and petroselinum sativum.

39. The composition of claim 27 which comprises substantially all natural and/or all organic ingredients and components.

40. The composition of claim 27 further comprising at least one component selected from material derived from an aloe vera plant, grapefruit seed extract and vitamin E.

41. The composition of claim 40 which comprises material derived from an aloe vera plant, grapefruit seed extract and vitamin E.

42. A personal lubricant composition comprising at least one component selected from the group consisting of lycopodium, testicle, sepia, hypericum, agnus castus, kidney, petroselinum sativum, liver, cantharis, berberis vulgaris and solidago vigaurea.

43. The composition of claim 42 which contains at least two components.

44. The composition of claim 42 which contains at least three components.

45. The composition of claim 42 which contains at least four components.

46. The composition of claim 42 which contains at least five components.

47. The composition of claim 42 which contains at least six components.

48. The composition of claim 42 which contains at least seven components.

49. The composition of claim 42 which contains at least eight components.

50. The composition of claim 42 which contains at least nine components.

51. The composition of claim 42 which contains at least ten components.

52. The composition of claim 42 which contains at least eleven two components.

53. The composition of claim 42 which comprises at least one component selected from the group consisting of lycopodium, testicle, sepia, agnus castus and petroselinum sativum.

54. The composition of claim 42 which comprises substantially all natural and/or all organic ingredients and components.

55. The composition of any of claims 1-54 wherein the strength of the homeopathic components used is lycopodium 6c, testicle 4c, sepia 6c, hypericum 6c, agnus castus 6c, kidney 9c, petroselinum sativum 6c, liver 9c, cantharis 6c, berberis vulgaris 6c and solidago vigaurea 6c.

56. A method of using the personal lubricant compositions of any of claims 1-55, comprising applying the lubricant to a male individual prior to sexual intercourse.
